(19) United States
(12) Patent Application Publication
(10) Pub. No.: US 2005/0129660 A1
(43) Pub. Date: Jun. 16, 2005

(54) Process of delivering a virally encapsulated polynucleotide or viral vector to a parenchymal cell via the vascular system

(76) Inventors: James E. Hagstrom, Middleton, WI (US); Jon A. Wolff, Madison, WI (US); Vladimir G. Budker, Middleton, WI (US); Julia Hegge, Monona, WI (US); Hans Herweijer, Madison, WI (US)

Correspondence Address: Mark K. Johnson;Mirus Corporation, 505 S. Rosa Rd., Madison, WI, 53719, US

(21) Appl. No.: 10/733,706

(22) Filed: Dec. 11, 2003

(51) Int. Cl. A61K 48/00; C12N 15/861; A61M 31/00

(52) U.S. Cl. 424/93.2; 435/456; 604/500

(57) ABSTRACT

A process for delivering a polynucleotide into a parenchymal cell in a mammal, comprising, transporting the polynucleotide into a vessel communicating with the parenchymal cell in tissue or organ of the mammal such that the polynucleotide is transfected into the parenchymal cell. More specifically, the polynucleotide consists of a viral vector.

### FIELD OF THE INVENTION

The invention generally relates to techniques for transferring genes into mammalian parenchymal cells in vivo. More particularly, a method is provided for transfecting parenchymal cells with viral encapsulated polynucleotides and viral vectors delivered intravascularly.

### BACKGROUND OF THE INVENTION

It was first observed that the in vivo injection of plasmid DNA directly into muscle tissue enabled the expression of foreign genes in the muscle [Wolff et al. 1990]. Since that report, several other studies have reported the ability for foreign gene expression following the direct injection of DNA into the parenchyma of other tissues. Naked DNA was expressed following its injection into cardiac muscle [Acsadi et al. 1991], pig epidermis [Hengge et al. 1995, rabbit thyroid [Sikes et al. 1994], lung by intratracheal injection [Meyer et al. 1995], into arteries using a hydrogel-coated angioplasty balloon [Riessen et al. 1993, Chapman et al. 1992], melanoma tumors [Vile et al. 1993] and rat liver [Malone et al. 1994, Hickman et al. 1994].

Another important target tissue for gene therapy is the mammalian liver, given its central role in metabolism and the production of serum proteins. A variety of techniques have been developed to transfer genes into the liver. Cultured hepatocytes have been genetically modified by retroviral vectors [Wolff et al. 1987, Ledley et al. 1987] and re-implanted into the livers in animals and in people [Chowdhury et al. 1991, Grossman et al. 1994]. Retroviral vectors have also been delivered directly to livers in which hepatocyte division was induced by partial hepatectomy [Kay et al. 1992, Ferry et al. PNAS; 1991, Kaleko et al. 1991]. The injection of adenoviral vectors into the portal or systemic circulatory systems leads to high levels of foreign gene expression that is transient [Stratford-Perricaudet et al. 1990, Jaffe et al. 1992, Li et al. 1993].

Foreign gene expression has also been achieved by repetitively injecting naked DNA in isotonic solutions into the liver parenchyma of animals treated with dexamethasone [Malone et al. 1994, Hickman et al. 1994]. Limited plasmid DNA expression in the liver has also been achieved via liposomes delivered by tail vein or intraportal routes [Kaneda et al. 1989, Soriano et al. 1983, Kaneda et al. 1989].

Despite this progress, there is still a need for a gene transfer method that can efficiently and safely cause the expression of foreign genes in parenchyma cells in vivo.

### SUMMARY OF THE INVENTION

The present invention provides for the transfer of polynucleotides into parenchymal cells within tissues in situ and in vivo. An intravascular route of administration enables a viral vector, virally encapsulated polynucleotide or a virally associated polynucleotide to be more evenly distributed to the parenchymal cells and expressed more efficiently than direct parenchymal injections. The efficiency of polynucleotide and viral vector delivery and expression is increased by increasing the permeability of vessels to the delivery vector. A volume is injected into the lumen of a vessel at an appropriate rate thereby increasing movement of the molecule or complex out of vessels and into the extravascular space. Increasing vessel permeability may further comprise blocking the flow of fluid through vessels into and/or out of a target tissue or area, increasing the intravascular hydrostatic (physical) pressure, and/or increasing the osmotic pressure.

In a preferred embodiment, we describe a method for delivering a polynucleotide to an organ or tissue cell, comprising: injecting a viral vector, virally encapsulated polynucleotide or a virally associated polynucleotide in a solution into the lumen of an afferent or efferent vessel of the organ or tissue. The method may further comprise occluding one or more afferent and/or efferent vessels of the organ or tissue. Occlusion of vessels facilitates increasing the volume of fluid in the target tissue or organ when the solution is injected.

In a preferred embodiment, we describe a method for delivering a polynucleotide to a muscle cell, comprising: inserting the polynucleotide into an efferent or afferent vessel of the tissue communicating with the muscle cell of the mammal such that the polynucleotide is transfected into the parenchymal cell.

A process is described for delivering a polynucleotide to a parenchymal cell of a mammal for expression of a gene, comprising, transporting the polynucleotide to a vessel containing a fluid; and, increasing the hydrostatic and/or osmotic pressure against the vessel wall for a time sufficient to complete delivery of the polynucleotide.

In a preferred embodiment, a process is described for increasing the transit of a viral vector, virally encapsulated polynucleotide or a virally associated polynucleotide out of a vessel and into a surrounding tissue in a mammal in vivo comprising: injecting a sufficient volume of injection solution containing the viral vector, virally encapsulated polynucleotide or a virally associated polynucleotide into an afferent or efferent vessel of the target tissue, thus forcing fluid out of the vasculature into the extravascular space. For injection into an artery, the target tissue is the tissue that the artery supplies with blood. For injection into a vein, the target tissue is the tissue from which the vein drains blood. For injection into bile duct, the target tissue is the liver. The injection solution may further contain a compound or compounds which may aid in delivery and may or may not associate with the molecule or complex.

In a preferred embodiment, the permeability of the vessel may be further increased by delivering to the mammal a compound which is known in the art to increase vessel permeability. Such compounds may be selected from the list comprising: histamine, vascular permeability factor, calcium channel blockers, beta-blockers, phorbol esters, ethylene-diaminetetraacetic acid, adenosine, papaverine, atropine, nifedipine, and hypertonic solutions.

In a preferred embodiment, the described devices and processes can be used to deliver a viral vector, virally encapsulated polynucleotide or a virally associated polynucleotide to a mammalian cell for the purpose of altering the endogenous properties of the cell. Altering the endogenous properties of the cell may be for therapeutic purposes, for facilitating pharmaceutical drug discovery, for facilitating drug target validation or for biological research. The mammal can be selected from the group comprising: mouse, rat, rabbit, guinea pig, dog, pig, goat, sheep, cow, primate and human. The cell may be selected from the group comprising parenchymal cell, liver cell, spleen cell, heart cell, kidney cell, lung cell, skeletal muscle cell, diaphragm cell, prostate cell, skin cell, testis cell, fat cell, bladder cell, brain cell, pancreas cell, and thymus cell.

Further objects, features, and advantages of the invention will be apparent from the following detailed description.

### BRIEF DESCRIPTION OF THE DRAWINGS

FIG. 1. Cross section of rat muscle (upper leg medial) following intra-arterial injection of adenovirus containing the β-galactosidase gene and β-galactosidase staining (100× magnification).

FIG. 2. Cross section of rat muscle (lower leg posterior) following intra-arterial injection of adenovirus containing the β-galactosidase gene and β-galactosidase staining, lower expression area (100× magnification).

### DETAILED DESCRIPTION OF THE INVENTION

We have developed an intravascular process for the delivery of viral vectors, virally encapsulated polynucleotides or a virally associated polynucleotides to extravascular parenchymal cells. A key advancement is the enhanced delivery to a larger and more even distribution of cells than is achieved using current gene delivery techniques. Furthermore, using this process, we show delivery of cationic, anionic and charge neutral macromolecules and complexes to tissue cells outside a vessel following injection into the lumen of the vessel. More efficient delivery is achieved by increasing permeability of vessels and increasing the volume of extravascular fluid in the target tissue in a target area. Vessel permeability is increased by one or more of the following: inserting a sufficient volume of an appropriate injection solution containing the molecule into the vessel, inserting the solution into the vessel at an appropriate rate, impeding fluid flow into and out of the target tissue during the process, and increasing permeability of the vessel wall.

Many blood vessels naturally contain pores or fenestrae to allow passage of nutrients, etc. However, in most tissues these pores are too small—about 4 nm diameter—to allow extravasation of many potentially therapeutic molecules including viral vectors. In addition, some potentially therapeutic molecules or vectors have poor biodistribution because of electrostatic interactions with serum components. Using the described processes, extravasation of fluid and viral vectors, virally encapsulated polynucleotides or a virally associated polynucleotides out of vessels and delivery to cells of the surrounding parenchyma is increased.

The term deliver means that the molecule or complex becomes associated with the cell thereby altering the endogenous properties of the cell. The molecule or complex can be on the membrane of the cell or inside the cytoplasm, nucleus, or other organelle of the cell. Other terms sometimes used interchangeably with deliver include transfect, transfer, or transform. In vivo delivery of a molecule or complex means to transfer the molecule or complex from a container outside a mammal to near or within the outer cell membrane of a cell in the mammal. The delivery of a biologically active compound is commonly known as “drug delivery”. A delivery system is the means by which a biologically active compound becomes delivered. The term encompasses all compounds, including the biologically active compound itself, and all processes required for delivery including the form and method of administration.

The described delivery system comprises an intravascular administration route. Vessels comprise internal hollow tubular structures connected to a tissue or organ within the body of an animal, including a mammal. Bodily fluid flows to or from the body part within the lumen of the tubular structure. Examples of bodily fluid include blood, lymphatic fluid, and bile. Vessels may be selected from the group comprising: arteries, arterioles, capillaries, venules, sinusoids, veins (including peripheral veins), lymphatics, and bile ducts. Afferent vessels are directed towards the organ or tissue and through which fluid flows towards the organ or tissue under normal physiological conditions. Conversely, efferent vessels are directed away from the organ or tissue and through which fluid flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. A vascular network consists of the directly connecting vessels supplying and/or draining fluid in a target organ or tissue.

An injector, such as a needle or catheter, is used to inject the viral vector, virally encapsulated polynucleotide or a virally associated polynucleotide into the vascular system. The injection can be performed under direct observation following an incision and visualization of the tissues blood vessels. Alternatively, a catheter can be inserted at a distant site and threaded so that it resides in the vascular system that connects with the target tissue. The injection can also be performed using a needle that traverses the intact skin and enters the lumen of a vessel. The viral vector, virally encapsulated polynucleotide or a virally associated polynucleotide can be injected into a blood vessel at a distal or proximal point. The viral vector, virally encapsulated polynucleotide or a virally associated polynucleotide can also be injected into a peripheral vein.

For some target tissues, the injection solution can be injected into either an afferent vessel or an efferent vessel. For example, for delivery to the liver, the injection solution can be inserted into the hepatic artery or the portal vein or via retrograde injection into the hepatic vein. Similarly, for delivery to heart muscle cells, the injection solution can be inserted into either arteries or veins. For injection into veins of skeletal muscle, the solution is injected in the direction of normal (antegrade) flow rather than in a retrograde direction.

Efficient delivery via intravascular administration primarily depends on the volume of the injection solution and the injection rate. Vessel occlusion is also an important factor for delivery to many tissues. The composition of the injection solution can depend on the nature of the molecule or complex that is to be delivered. We have observed that certain complexes may be delivered more efficiently using low salt injection solutions. The use or hypertonic or hypotonic injection solutions or the use of vasodilators in the injection solution may further enhance delivery.

The choice of injection volume and rate are dependent upon: the size of the animal, the size of the vessel into which the solution is injected, the size and or volume of the target tissue, the bed volume of the target tissue vasculature, and the nature of the target tissue or vessels supplying the target tissue. For example, delivery to liver may require less volume because of the porous nature of the liver vasculature. The precise volume and rate of injection into a particular vessel, for delivery to a particular target tissue, may be determined empirically. Larger injection volumes and/or higher injection rates are typically required for a larger vessels, target sizes, etc. For example, efficient delivery to mouse liver may require injection of as little as 1 ml or less (animal weight ˜25 g). In comparison, efficient delivery to dog or nonhuman primate limb muscle may require as much as 60-500 ml or more (animal weight 3-14 kg). Injection rates can vary from 0.5 ml/sec or lower to 4 ml/sec or higher, depending on animal size, vessel size, etc. Occlusion of vessels, by balloon catheters, clamps, cuffs, natural occlusion, etc, can limit or define the vascular network size or target area.

Because vasculature may not be identical from one individual to another, methods may be employed to predict or control appropriate injection volume and rate. Injection of iodinated contrast dye detected by fluoroscopy can aid in determining vascular bed size. Also, an automatic injection system can be used such that the injection solution is delivered at a preset pressure. For such a system, pressure may be measured in the injection apparatus, in the vessel into which the solution is injected, in a branch vessel within the target tissue, or within an efferent or afferent vessel within the target tissue.

Injecting into a vessel an appropriate volume at an appropriate rate increases the volume of fluid in the tissue while increasing permeability of the vessel to the injection solution and the molecules or complexes therein. Permeability can be further increased by occluding outflow of fluid (both bodily fluid and injection solution) from the tissue or local vascular network. For example, a solution is rapidly injected into an afferent vessel supplying an organ while the efferent vessel(s) draining the tissue is transiently occluded. Branching vessels may also be occluded. Natural occlusions may also be used. The afferent vessel may also be transiently occluded proximal to the injection site. The vessels are partially or totally occluded for a period of time sufficient to allow delivery of a molecule or complex present in the injection solution. The occlusion may be released immediately after injection or may be released only after a determined length of time which does not result in tissue damage due to ischemia. Permeability is defined herein as the propensity for macromolecules to move out of a vessel and enter the extravascular space. One measure of permeability is the rate at which macromolecules move through the vessel wall and out of the vessel. Another measure of permeability is the lack of force that resists the movement of fluid or macromolecules through the vessel wall and out of the vessel. Endothelial cells lining the interior of blood vessels and connective material (e.g., collagen) both function to limit permeability of blood vessels to macromolecules. Increasing the size of the tissue is defined herein as increasing extracellular volume and/or cell volume in the specific tissue.

One method for occluding fluid flow through vessels is the application of an external cuff. The term cuff means an device for impeding fluid flow through mammalian vessels, particularly blood vessels. More particularly, a cuff refers specifically to a device applied exterior to the mammal's skin that touches the skin in a non-invasive manner. The cuff applies external compression to the mammalian skin such that vessel walls, in an area underneath the cuff, are forced to constrict an amount sufficient to impede fluid from flowing through the vessels at a normal rate. Impeding fluid flow into and out of an area such as a limb, combined with injection of a solution, causes increased vessel permeability to increase the size and volume of the tissue. One example of a cuff is a sphygmomanometer which is normally used to measure blood pressure. Another example is a tourniquet. An exterior cuff may be applied prior to insertion of the injection solution, subsequent to insertion, or concurrent with insertion.

The described intra-arterial and intravenous processes require that blood flow be impeded for substantially less time than is required to cause tissue damage by ischemia. In fact, a common anesthesia for human limb surgery (e.g., carpal tunnel repair) involves the blockage of blood flow for more than one hour. We have not observed any widespread histological evidence of ischemic muscle damage in mice, rats, dogs, or primates following the described processes. The minimal elevations of muscle-derived enzymes found in serum provide significant evidence against any consequential muscle damage.

These techniques may be combined with other agents, vasodilators, known in the art for increasing vascular permeability, including drugs or chemicals and hypertonic solutions. Drugs or chemicals can increase the permeability of the vessel by causing a change in function, activity, or shape of cells within the vessel wall; typically interacting with a specific receptor, enzyme or protein of the vascular cell. Other agents can increase permeability by changing the extracellular connective material. Examples of drugs or chemicals that may be used to increase vessel permeability include histamine, vascular permeability factor (VPF, which is also known as vascular endothelial growth factor, VEGF), calcium channel blockers (e.g., verapamil, nicardipine, diltiazem), beta-blockers (e.g., lisinopril), phorbol esters (e.g., PKC), ethylenediaminetetraacetic acid (EDTA), adenosine, papaverine, atropine, and nifedipine. Hypertonic solutions have increased osmolarity compared to the osmolarity of blood thus increasing osmotic pressure and causing cells to shrink. Typically, hypertonic solutions containing salts such as NaCI or sugars or polyols such as mannitol are used.

Molecules and complexes can be efficiently delivered to skeletal muscle cells in vivo via intravascular delivery. For example, up to 21% of all muscle cells in rat hind limbs express β-galactosidase after injection of 500 μg pCI-LacZ plasmid DNA in 10 ml saline into the iliac artery [Zhang et al. 2001]. Similar experiments in pig heart demonstrated that cardiac tissue can be efficiently transfected following injection of 1.5 mg plasmid DNA in 30 ml saline. Delivery of plasmid DNA to heart muscle cells, as determined by luciferase expression, is equally efficient following injection into coronary arteries or veins.

In the heart, efficient delivery through a coronary vein does not require occluding fluid flow through the corresponding artery. In this case, the microcapillary bed generates sufficient resistance to increased vessel permeability following solution injection. In ischemic heart, pre-existing artery blockages may help to increase delivery by occluding fluid flow through the artery. For insertion of the injection solution, percutaneous transluminal coronary angioplasty (PTCA) catheters may be advanced into the coronary venous system from a peripheral vein.

Delivery to the liver by injection into the hepatic vein is an example of retrograde delivery. As demonstrated in the examples that follow, injections can be directed into the inferior cava which is occluded both proximally and distally to the entry of the hepatic vein into the inferior vena cava. Specifically, the downstream inferior vena cava occlusion is placed between the diaphragm and the entry point of the hepatic vein. The upstream inferior vena cava occlusion is placed just upstream of the entry point of the renal veins. The hepatic artery, mesenteric artery, renal vein and portal vein can also be occluded.

It may be beneficial for multiple vessels connecting to a single target tissue to be injected, either simultaneously or sequentially. For example, for delivery to liver, injections solutions may be inserted into both the bile duct and the portal vein.

It is envisioned that the described processes may be used repetitively in a single mammal. Multiple injections may be used to provide delivery to additional tissues, to increase delivery to a single tissue, or where multiple treatments are indicated, or to facilitate longer term expression.

The processes are shown to be effective in mice, rats, dogs, pig, and non-human primates. That delivery is observed in each of these animals strongly suggests that the processes are generally applicable to all mammals. In particular, the effectiveness of the processes in delivering molecules and complexes to nonhuman primates indicates that the processes will also be successful in humans.

The described processes may be combined with other delivery vehicles or vectors or other delivery enhancing groups. Such delivery vehicles and groups comprise: transfection reagents, “naked” plasmid DNA, siRNA, non-viral vectors, lipids, polymers, polycations, amphipathic compounds, targeting signals, nuclear targeting signals, and membrane active compounds.

Delivery may also be improved by the use of tissue specific cellular targeting signals; enhance binding to receptors, cytoplasmic transport to the nucleus and nuclear entry (nuclear localizing signals) or release from endosomes or other intracellular vesicles. Cellular receptor signals are any signal that enhances the association of the gene with a cell, including ligands and non-specific cell binding. A targeting signal can be a protein, peptide, lipid, steroid, sugar, carbohydrate, nucleic acid or synthetic compound.

### Definitions

The term polynucleotide is a term of art that refers to a string of at least two (nucleotide) base-sugar-phosphate combinations. Nucleotides are the monomeric units of nucleic acid polymers. The term includes deoxyribonucleic acid (DNA) and ribonucleic acid (RNA) in the form of an oligonucleotide messenger RNA, anti-sense, plasmid DNA, parts of a plasmid DNA or genetic material derived from a virus. Anti-sense is a polynucleotide that interferes with the function of DNA and/or RNA.

A polynucleotide can be delivered to a cell in order to produce a cellular change that is therapeutic or furthers other research purposes. The delivery of polynucleotides or other genetic material for therapeutic(the art of improving health in an animal including treatment or prevention of disease) and/or research purposes is gene therapy. The polynucleotides are coded to express a whole or partial protein, or may be or encode anti-sense or other functional nucleic acid (i.e. siRNA). The protein can be missing or defective in an organism as a result of genetic, inherited or acquired defect in its genome. For example, a polynucleotide may be coded to express the protein dystrophin that is missing or defective in Duchenne muscular dystrophy. The coded polynucleotide is delivered to a selected group or groups of cells and incorporated into those cell's genome or remain apart from the cell's genome. Subsequently, protein expressed from the polynucleotide is produced by the formerly deficient cells. Other examples of imperfect protein production that can be treated with gene therapy include the addition of the protein clotting factors that are missing in the hemophilias and enzymes that are defective in inborn errors of metabolism such as phenylalanine hydroxylase. A delivered polynucleotide can also be therapeutic in acquired disorders such as neurodegenerative disorders, cancer, heart disease, and infections. The polynucleotide has its therapeutic effect by entering the cell. Entry into the cell is required for the polynucleotide to produce the therapeutic protein, to block the production of a protein, or to decrease the amount of a RNA.

Duchenne Delivery of a polynucleotide means to transfer a polynucleotide from a container outside a mammal to within or near the outer cell membrane of a cell in the mammal. The term transfection is used herein, in general, as a substitute for the term delivery, or, more specifically, the transfer of a polynucleotide from outside a cell membrane to within the cell membrane. If the polynucleotide is a primary RNA transcript that is processed into messenger RNA, a ribosome translates the messenger RNA to produce a protein within the cytoplasm. If the polynucleotide is a DNA, it enters the nucleus where it is transcribed into a messenger RNA that is transported into the cytoplasm where it is translated into a protein. The polynucleotide may contain sequences that are required for transcription and translation. These sequences may include promoter and enhancer sequences that are required for initiation. DNA and thus the corresponding messenger RNA (transcribed from the DNA) may contain introns, poly A sequences, and sequences required for the initiation and termination of its translation into protein. Therefore if a polynucleotide expresses its cognate protein, then it must have entered a cell.

A therapeutic effect of the protein in attenuating or preventing the disease state can be accomplished by the protein either staying within the cell, remaining attached to the cell in the membrane or being secreted and dissociating from the cell where it can enter the general circulation and blood. Secreted proteins that can be therapeutic include hormones, cytokines, growth factors, clotting factors, anti-protease proteins (e.g. alpha-antitrypsin) and other proteins that are present in the blood. Proteins on the membrane can have a therapeutic effect by providing a receptor for the cell to take up a protein or lipoprotein. For example, the low density lipoprotein (LDL) receptor could be expressed in hepatocytes and lower blood cholesterol levels and thereby prevent atherosclerotic lesions that can cause strokes or myocardial infarction. Therapeutic proteins that stay within the cell can be enzymes that clear a circulating toxic metabolite as in phenylketonuria. They can also cause a cancer cell to be less proliferative or cancerous (e.g. less metastatic). A protein within a cell could also interfere with the replication of a virus.

A therapeutic effect of an siRNA or oligonucleotide in attenuating or preventing an unwanted cellular state can be accomplished by the siRNA or oligonucleotide entering the cell and acting on messenger RNA in the cytoplasm or nucleus, or by an oligonucleotide acting on genomic DNA or precursor RNAs within the nucleus of a cell.

The delivered polynucleotide can stay within the cytoplasm or nucleus apart from the endogenous genetic material. Alternatively, the polynucleotide could recombine (become a part of) with the endogenous genetic material. For example, DNA can insert into chromosomal DNA by either homologous or non-homologous recombination.

Parenchymal cells are the distinguishing cells of a gland or organ contained in and supported by the connective tissue framework. The parenchymal cells perform a function that is unique to the particular organ. The term “parenchymal” excludes cells that are common to many organs and tissues such as fibroblasts and endothelial cells within the blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and apposed sides to a bile canaliculus. Liver cells that are not parenchymal cells include cells within the blood vessels such as the endothelial cells or fibroblast cells.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

In a pancreas, the parenchymal cells include cells within the acini such as zymogenic cells, centroacinar cells, and basal or basket cells and cells within the islets of Langerhans such as alpha and beta cells.

In spleen, thymus, lymph nodes and bone marrow, the parenchymal cells include reticular cells and blood cells (or precursors to blood cells) such as lymphocytes, monocytes, plasma cells and macrophages.

In the nervous system which includes the central nervous system (the brain and spinal cord) peripheral nerves, and ganglia, the parenchymal cells include neurons, glial cells, microglial cells, oligodendrocytes, Schwann cells, and epithelial cells of the choroid plexus.

In the kidney, parenchymal cells include cells of collecting tubules and the proximal and distal tubular cells. In the prostate, the parenchyma includes epithelial cells.

In glandular tissues and organs, the parenchymal cells include cells that produce hormones. In the parathyroid glands, the parenchymal cells include the principal cells (chief cells) and oxyphilic cells. In the thyroid gland, the parenchymal cells include follicular epithelial cells and parafollicular cells. In the adrenal glands, the parenchymal cells include the epithelial cells within the adrenal cortex and the polyhedral cells within the adrenal medulla.

In the parenchyma of the gastrointestinal tract such as the esophagus, stomach, and intestines, the parenchymal cells include epithelial cells, glandular cells, basal, and goblet cells.

In the parenchyma of lung, the parenchymal cells include the epithelial cells, mucus cells, goblet cells, and alveolar cells.

In fat tissue, the parenchymal cells include adipose cells or adipocytes. In the skin, the parenchymal cells include the epithelial cells of the epidermis, melanocytes, cells of the sweat glands, and cells of the hair root.

In cartilage, the parenchyma includes chondrocytes. In bone, the parenchyma includes osteoblasts, osteocytes, and osteoclasts.

An intravascular route of administration enables a viral vector and/or a virally encapsulated polynucleotide to be delivered to parenchymal cells more evenly distributed and more efficiently expressed than direct parenchymal injections.

Polypeptide refers to a linear series of amino acid residues connected to one another by peptide bonds between the alpha-amino group and carboxy group of contiguous amino acid residues.

Vectors include polynucleic molecules originating from a virus, a plasmid, or the cell of an organism into which another nucleic fragment of appropriate size can be integrated without loss of the vectors capacity for self-replication; vectors introduce foreign DNA into host cells, where it can be reproduced. Examples are plasmids, cosmids, yeast artificial chromosomes and viruses. Vectors are often recombinant molecules containing DNA sequences from several sources. A vector includes a viral vector selected from the list comprising: adeno-associated virus (Parvoviridae), adenovirus (icosahedral virus that contains DNA; there are over 40 different adenovirus varieties, some of which cause the common cold), herpes simplex virus (HSV), vaccinia virus (Poxviridae), retrovirus (Retroviridae), murine leukaemia virus, lentivirus, human immunodeficiency virus (HW), syndbis virus (Togaviridae), vesicular stomatitis virus (VSV, Rhabdoviridae) and recombinant virus.

Afferent blood vessels of organs are defined as vessels which are directed towards the organ or tissue and in which blood flows towards the organ or tissue under normal physiologic conditions. Conversely, the efferent blood vessels of organs are defined as vessels which are directed away from the organ or tissue and in which blood flows away from the organ or tissue under normal physiologic conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. A liver blood vessel includes the portal venous system which transports blood from the gastrointestinal tract and other internal organs (e.g. spleen, pancreas and gall bladder) to the liver. Another liver blood vessel is the hepatic vein. The hepatic vein may also be reached via the inferior vena cava or another blood vessel that ultimately connects to the liver.

### EXAMPLES

### Example 1

Adenoviral Vectors can be Delivered to Muscle Parenchymal Cells by an Intravascular Route.

Adult Sprague-Dawley rats (120-140 g) were anesthetized with isoflorane and the surgical field was shaved and prepped with an antiseptic. The animals were placed on a heating pad to prevent loss of body heat during the surgical procedure. A 4 cm long abdominal midline incision was made after which skin flaps were folded away and held with clamps to expose the target area. A moist gauze was applied to prevent excessive drying of internal organs. Intestines were moved to visualize the iliac veins and arteries. Microvessel clips were placed on the external iliac, caudal epigastric, internal iliac, deferent duct, and gluteal arteries and veins as well as on the inferior vena cava near the bifurcation to block both outflow and inflow of the blood to the leg. An efflux enhancer solution (e.g., 0.5 mg of papaverine and 40 ng of collagenase in 3 ml saline) was injected into the external iliac artery though a 25 g needle. 1-10 minutes later, a 27 G butterfly needle was inserted into the external iliac artery and 10 ml normal saline containing 5×10⁸ Adenovirus CMVLuc particles was injected in about 10 sec. Fluid was injected in the direction of normal blood flow. The adenoviral vector CMVLuc expresses the luciferase gene from the immediate early promoter of the human cytomegalovirus [Yang T et al. 1996]. The microvessel clips were removed 2 minutes after the injection and bleeding was controlled with pressure and gel foam. The abdominal muscles and skin were closed with 4-0 dexon suture. Two days after injection the leg muscle were assayed for luciferase as above.

Luciferase Assays: Results of the rat AdV-Luc injections are provided in relative light units (RLU) and/or micrograms (μg) of luciferase produced. To determine RLU, 10 μl of cell lysate were assayed using a EG&G Berthold LB9507 luminometer and total muscle RLU were determined by multiplying by the appropriate dilution factor.

TABLE 1
Distribution of luciferase activity following the intraarterial injection
of adenovirus CMVLuc.

| Muscle Group | Luciferase (ng) |
|---|---|
| Upper Leg Anterior | 59.04 |
| Upper Leg Posterior | 18.33 |
| Upper Leg Medial | 4.44 |
| Lower Leg Posterior | 11.04 |
| Lower Leg Anterior | 5.33 |
| Foot | 0.22 |
| Total | 98.40 |

The increased permeability of vessels resulting form the injection procedure enabled delivery of adenovirus to muscles cells in the leg and expression of the adenovirus encoded luciferase gene.

### Example 2

rAAV CMV-Luciferase Delivery Protocol

Recombinant AAV viral particles containing reporter genes were delivered to muscles in the leg of a rat via a single intra-arterial injection and the resulting gene expression was determined. We compared the efficiency of single-site intravascular gene delivery system to that of multi-site direct intramuscular injection for the delivery of recombinant AAV (rAAV) to muscle. Recombinant AAV particles (AAV-Luciferase, AAV-LacZ, AAV-AAT) were injected into rat leg muscle by either a single intra-arterial injection into the external iliac as indicated above for delivery of adenovirus or by direct injections into each of 5 major muscle groups of the leg [see Wolff et al. 1990]. For direct intramuscular injections, 1×10¹² rAAV particles were split and equal amounts were injected into each of 5 muscle groups: upper leg anterior, upper leg posterior, upper leg medial, lower leg anterior, lower leg posterior. All rats used were female and approximately 150 grams and each received a total of 1×10¹² rAAV particles via injection. Luciferase of β-galactosidase expression in muscle cells was determined at various times after injection.

Luciferase Assays: Results of the rat AdV-Luc injections are provided in relative light units (RLU) and/or micrograms (μg) of luciferase produced. To determine RLU, 10 μl of cell lysate were assayed using a EG&G Berthold LB9507 luminometer and total muscle RLU were determined by multiplying by the appropriate dilution factor.

TABLE 2
Comparison of luciferase expression in skeletal muscle cells following
delivery of AAV by intravascular delivery or intramuscular injection.

| | |
|---|---|
| muscle group | relative light units (RLU) |

Intravascular (IV) Delivery - 2 week time point

| | | | | |
|---|---|---|---|---|
| upper leg | 1.80 × 10¹⁰ | 6.10 × 10¹⁰ | 6.37 × 10¹⁰ | 1.03 × 10¹⁰ |
| anterior | | | | |
| upper leg | 2.38 × 10¹⁰ | 5.17 × 10¹⁰ | 8.27 × 10⁹ | 4.62 × 10⁹ |
| posterior | | | | |
| upper leg | 1.12 × 10¹⁰ | 3.50 × 10¹⁰ | 9.29 × 10⁹ | 5.79 × 10⁹ |
| medial | | | | |
| lower leg | 1.30 × 10¹⁰ | 1.09 × 10¹¹ | 2.01 × 10¹⁰ | 8.32 × 10⁹ |
| posterior | | | | |
| lower leg | 4.32 × 10⁹ | 5.01 × 10⁹ | 1.29 × 10¹⁰ | 5.04 × 10⁹ |
| anterior | | | | |
| foot | 5.08 × 10⁸ | 9.51 × 10⁹ | 1.52 × 10⁸ | 8.20 × 10⁷ |
| total RLU | 7.08 × 10¹⁰ | 2.71 × 10¹¹ | 1.14 × 10¹¹ | 3.41 × 10¹⁰ |
| μg luciferase | 3.61 | 13.85 | 5.84 | 1.75 |

| | |
|---|---|
| average | 6.26 μg luciferase per leg |

Direct Intramuscular (IM) Injection - 2 week time point

| upper leg | 1.61 × 10¹⁰ | 1.21 × 10¹⁰ | 1.41 × 10¹⁰ | 3.41 × 10¹⁰ |
|---|---|---|---|---|
| anterior | | | | |
| upper leg | 3.34 × 10⁹ | 3.62 × 10⁹ | 4.49 × 10⁹ | 3.26 × 10⁹ |
| posterior | | | | |
| upper leg | 8.88 × 10⁹ | 1.75 × 10¹⁰ | 6.49 × 10⁹ | 1.41 × 10¹⁰ |
| medial | | | | |
| lower leg | 8.12 × 10⁹ | 1.03 × 10¹⁰ | 1.71 × 10¹⁰ | 7.73 × 10⁹ |
| posterior | | | | |
| lower leg | 0.23 × 10⁹ | 1.47 × 10¹⁰ | 3.57 × 10¹⁰ | 4.09 × 10¹⁰ |
| anterior | | | | |
| foot | 6.01 × 10⁶ | 5.23 × 10⁷ | 2.35 × 10⁶ | 2.26 × 10⁶ |
| total RLU | 3.67 × 10¹⁰ | 5.82 × 10¹⁰ | 7.79 × 10¹⁰ | 1.00 × 10¹¹ |
| μg luciferase | 1.86 | 2.97 | 3.97 | 5.11 |
| average | 3.48 μg luciferase per leg | | | |
| | IV delivery | | IM injection | |
| | 4 week time point | | 4 week time point | |
| upper leg | 1.03 × 10¹¹ | 1.69 × 10¹⁰ | 5.71 × 10¹⁰ | 4.21 × 10¹⁰ |
| anterior | | | | |
| upper leg | 6.65 × 10¹⁰ | 1.73 × 10¹⁰ | 3.96 × 10¹⁰ | 1.36 × 10¹⁰ |
| posterior | | | | |
| upper leg | 1.17 × 10¹¹ | 4.23 × 10¹⁰ | 1.86 × 10¹⁰ | 3.30 × 10¹⁰ |
| medial | | | | |
| lower leg | 2.15 × 10¹¹ | 6.09 × 10¹⁰ | 3.19 × 10¹⁰ | 2.46 × 10¹⁰ |
| posterior | | | | |
| lower leg | 1.62 × 10¹⁰ | 2.00 × 10¹⁰ | 3.62 × 10¹⁰ | 2.36 × 10¹⁰ |
| anterior | | | | |
| foot | 7.50 × 10⁹ | 0.36 × 10⁹ | 0.09 × 10⁹ | 0.05 × 10⁸ |
| total RLU | 5.25 × 10¹¹ | 1.58 × 10¹¹ | 1.84 × 10¹¹ | 1.37 × 10¹¹ |
| μg luciferase | 26.82 | 8.05 | 9.36 | 6.97 |

| | | |
|---|---|---|
| average | 17.44 μg luciferase per leg | 8.17 μg luciferase per leg |

| | IV delivery | | IM injection |
|---|---|---|---|
| | 8 week time point | | 8 week time point |
| upper leg anterior | 6.82 × 10¹⁰ | 4.03 × 10¹⁰ | 9.93 × 10¹⁰ |
| upper leg posterior | 2.22 × 10¹⁰ | 4.40 × 10⁹ | 3.13 × 10¹⁰ |
| upper leg medial | 4.98 × 10¹⁰ | 3.57 × 10¹⁰ | 1.89 × 10¹⁰ |
| lower leg posterior | 5.87 × 10¹⁰ | 7.56 × 10¹⁰ | 9.39 × 10¹⁰ |
| lower leg anterior | 2.29 × 10¹⁰ | 2.77 × 10¹⁰ | 8.35 × 10¹⁰ |
| foot | 0.7 × 10⁹ | 0.28 × 10⁹ | 0.10 × 10⁹ |
| total RLU | 2.23 × 10¹¹ | 1.84 × 10¹¹ | 3.27 × 10¹¹ |
| μg luciferase | 11.27 | 9.38 | 16.68 |

| | | |
|---|---|---|
| average | 10.32 μg luciferase per leg | 16.68 μg |
| | | luciferase per leg |

Delivery of rAAV CMV-Luc into rat muscle via the described intravascular delivery procedure results in high levels of transgene expression. Average total luciferase levels were higher for the single-site intra-arterial (10.07 μg per animal; n=8) method than for the multi-site direct muscle injections (6.70 μg per animal; n=7). High level luciferase expression is stable out to at least 8 weeks in rats with an intact immune system (Harlan Sprague Dawley rats)

### Example 3

rAAV CMV-LacZ Delivery:

To confirm that AAV mediated gene expression was occurring within muscle parenchyma, injections were performed using an AAV vector containing the reporter gene β-galactosidase. Recombinant AAV CMV-LacZ (1×10¹² particles) was injected into 7 animals (4 intra-arterial, 3 direct muscle injection) as described above. All animals tolerated the procedure well and muscle was harvested at 2, 4, 6 and 8 weeks for the intra-arterial delivery and 2, 4 and 8 weeks for the direct injections.

β-galactosidase Assays: Animals were euthanized at the indicated times. Following excision, muscles were cut into two pieces and frozen in liquid nitrogen cooled isopentane. Frozen muscles were embedded, sliced into thin sections on a cryostat (6-10 microns thick) and mounted onto glass slides. Mounted sections were fixed in glutaraldehyde and stained for β-galactosidase activity.

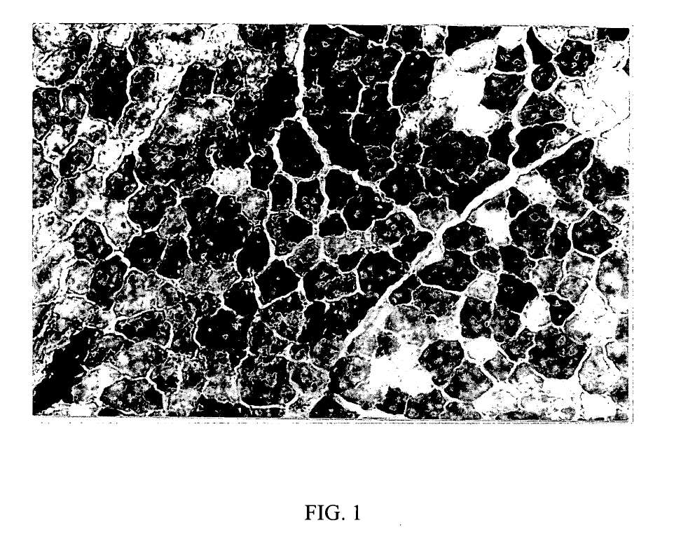

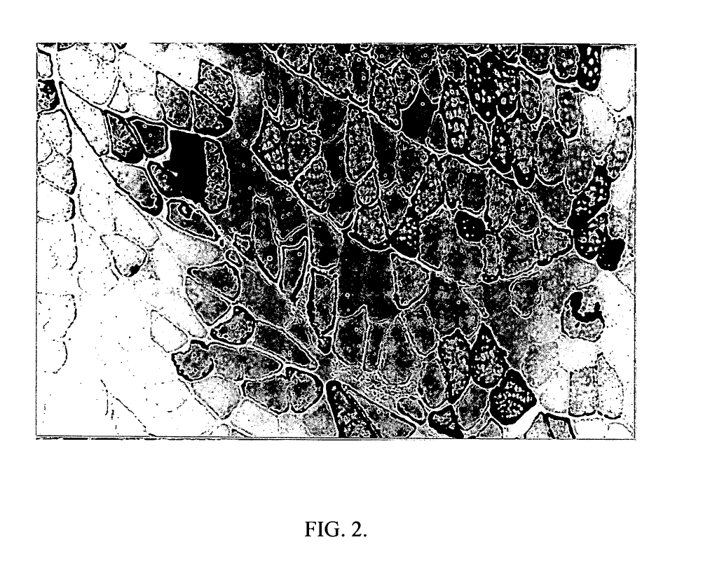

β-galactosidase staining of thin sections indicated that the LacZ reporter gene was efficiently expressed in myofibers (FIG. 1, upper leg medial, high expression area; FIG. 2, lower leg posterior, lower expression area). All muscles excised (from both intra-arterial and direct injection) displayed β-galactosidase expression within myofibers with the major difference being the distribution of the staining. In the direct muscle injection samples, β-galactosidase staining was localized to areas near the injection site as expected. In the rats receiving intra-arterial injections, myofiber expression was much more widespread with LacZ positive stained cells being found in all parts of the excised leg muscles. Expression of β-galactosidase in the rat muscle following intra-arterial injection was much more widespread than following direct injection.

Delivery of rAAV CMV-LacZ results in high levels of myofiber gene expression using both intra-arterial and direct muscle injection. A single intra-arterial (external iliac) injection of rAAV resulted in β-galactosidase expression in all major muscle groups in the rat leg. Expression patterns were qualitatively different between the two injection procedures with more widespread myofiber expression occurring in muscle groups receiving intra-arterial injections. Percent myofiber staining ranged from ˜2-5% (low expressing areas) to ˜100% blue cells (high expressing areas) for intra-arterial delivery and ˜0% (away from injection site) to ˜100% (near injection site) for direct muscle injection depending on location.

### Example 4

Delivery of Adenovirus to Limb Skeletal Muscle Via Venous Injection.

Delivery of Adenovirus and siRNA to limb muscle cells via saphenous vein injection: 120-140 g adult Sprague-Dawley rats were anesthetized with isoflurane and the surgical field was shaved and prepped with an antiseptic. The animals were placed on a heating pad to prevent loss of body heat during the surgical procedure. A latex tourniquet was wrapped around the upper limb and secured with a hemostat. A 1.5 cm incision was made on the inside of the limb to expose the medial saphenous vein. A 25-gauge needle catheter was inserted into the distal great saphenous vein and secured with a microvascular clip. The needle catheter was connected to a two-way connector for delivering both papaverine and pDNA and fluid was injected in the direction of normal blood flow. All animals were injected with 1.5 ml of papaverine (0.25 mg in saline) over 6 seconds using a syringe pump. After 5 min, 5 ml normal saline containing 2×10⁹ Adenovirus particles encoding firefly Luciferase was injected at varying flow rates. Some injections also contained 5 μg of the siRNA targeted against firefly luc⁺ (siRNA-luc⁺). 2 minutes after injection, the tourniquet and catheter were removed and the skin was closed with 4-0 Vicryl.

Delivery of Adenovirus and siRNA to limb muscle cells via direct muscular injection: 120-140 g adult Sprague-Dawley rat was anesthetized with isoflurane. The animals were placed on a heating pad to prevent loss of body heat during the procedure. 1×10⁹ Adenovirus particles encoding firefly Luciferase in 2.5 ml of saline was injected into each hind gastrocnemius muscle group of the animal.

TABLE 3
Delivery of adenovirus to skeletal muscle: intravenous vs. intramuscular injection.

| nucleic acid | | injection | | | luciferase activity | |
|---|---|---|---|---|---|---|
| Adenovirus | siRNA | Route | Volume (ml) | rate (ml/min) | total RLUs | ng per limb |
| 2.0 × 10⁹ | | saphenous | 5.0 | 16.6 | 1,613,367 | 101.8 |
| 2.0 × 10⁹ | | saphenous | 5.0 | 12.5 | 656,435 | 48.2 |
| 2.0 × 10⁹ | | saphenous | 5.0 | 12.5 | 222,172 | 16.2 |
| 2.0 × 10⁹ | 5 μg | saphenous | 5.0 | 16.6 | 112,325 | 7.3 |
| 2.0 × 10⁹ | 5 μg | saphenous | 5.0 | 16.6 | 101,824 | 5.4 |
| 1.0 × 10⁹ | | IM (gastroc) | 2.5 | | 3,262 | 0.2 |
| 1.0 × 10⁹ | | IM (gastroc) | 2.5 | | 2,724 | 0.2 |

Conclusion: Injection of adenovirus into a leg vein, using the describe delivery process, resulted in efficient delivery of the virus to muscle cells in the leg and expression of a virally encoded reporter gene. Delivery was much more efficient that direct muscle injections. Co-injection of siRNA specific to the luciferase gene resulted in efficient inhibition of luciferase expression

### Example 5

Delivery to Rat Skeletal Muscle Cells In Vivo Using Intra-Iliac Injection.

250 μg pCI-Luc plasmid DNA in 10 ml Ringer's injection solution was injected into iliac artery of rats using a Harvard Apparatus PHD 2000 programmable syringe pump. Varying injection rates were used. Specifically, animals were anesthetized and the surgical field shaved and prepped with an antiseptic. The animals were placed on a heating pad to prevent loss of body heat during the surgical procedure. A midline abdominal incision was be made after which skin flaps were folded away and held with clamps to expose the target area. A moist gauze was applied to prevent excessive drying of internal organs. Intestines were moved to visualize the iliac veins and arteries. Microvessel clips were placed on the external iliac, caudal epigastric, internal iliac, deferent duct, and gluteal arteries and veins to block both outflow and inflow of the blood to the leg. An efflux enhancer solution (e.g., 0.5 mg papaverine in 3 ml saline) was pre-injected into the external iliac artery though a 25 g needle. Ten min later, 12 mL injection solution containing the indicated complexes was injected in approximately 10 seconds. The microvessel clips were removed 2 minutes after the injection and bleeding was controlled with pressure and gel foam. The abdominal muscles and skin were closed with 4-0 dexon suture. Seven days after injection, the animals were sacrificed, and a luciferase assays were conducted on leg muscles. Results show that efficiency of delivery is affected by the rate of solution injection.

TABLE 4
Luciferase expression (ng Luciferase) after delivery of plasmid DNA
to muscle via iliac administration route.

| | Injection Rate | | | |
|---|---|---|---|---|
| | 0.83 ml/sec | 0.56 ml/sec | 0.42 ml/sec | 0.33 ml/sec |
| muscle | n = 2 | n = 4 | n = 3 | n = 3 |
| quad | 1109 ± 1183 | 384 ± 386 | 733 ± 154 | 221 ± 246 |
| biceps | 1476 ± 1138 | 276 ± 185 | 604 ± 122 | 83 ± 37 |
| hamstring | 2413 ± 1045 | 2071 ± 942 | 1635 ± 643 | 706 ± 384 |
| gastrocnemius | 1852 ± 1316 | 2274 ± 673 | 2088 ± 329 | 1078 ± 372 |
| shin | 774 ± 610 | 367 ± 361 | 289 ± 274 | 189 ± 63 |
| foot | 6 ± 5.5 | 8.9 ± 10.7 | 4.3 ± 2.2 | 0.9 ± 0.2 |
| total | 7397 ± 4456 | 7389 ± 2062 | 6664 ± 1001 | 3338 ± 1762 |

### Example 6

Delivery of Polynucleotides to Liver in Mouse: Comparison of Ringer's and Low-Salt Glucose Injection Solutions for Delivery by Peripheral Vein (Tail Vein) Injections.

Two solutions were used in this experiment. Solution A was prepared consisting of 290 mM glucose, 5 mM Hepes, adjusted to pH 7.5. Solution B was Ringer's.

Complexes were prepared as follows:
Complex I. pDNA (45 μg, 22.5 μL of a 2 μg/μL solution in water) was added to 11.25 mL of Solution A.
Complex II. pDNA (45 μg, 22.5μL of a 2 μg/μL solution in water) was added to 11.25 mL of Solution A. To this solution was added Histone H1 (270 μg, 27 μL of a 10 mg/mL solution in water), and the sample was mixed.
Complex III. pDNA (45 μg, 22.5 μL of a 2 μg/μL solution in water) was added to 11.25 mL of Solution A. To this solution was added Histone H1 (50 μg, 5 μL of a 10 mg/mL solution in water), and the sample was mixed.
Complex IV. pDNA (45 μg, 22.5 μL of a 2 μg/μL solution in water) was added to 11.25 mL of Solution A. To this solution was added Histone H1 (36 82 g, 3.6 μL of a 10 mg/mL solution in water), and the sample was mixed.
Complex V. pDNA (45 μg, 22.5 μL of a 2 μg/μL solution in water) was added to 11.25 mL of Solution B.
Complex VI. pDNA (45 μg, 22.5 μLg of a 2 μg/μL solution in water) was added to 11.25 mL of Solution B. To this solution was added Histone H1 (270 μg, 27 μL of a 10 mg/mL solution in water), and the sample was mixed.
Complex VII. pDNA (45 μg, 22.5 μL of a 2 μg/μL solution in water) was added to 11.25 mL of Solution B. To this solution was added Histone H1 (50 μg, 5 μL of a 10 mg/mL solution in water), and the sample was mixed.
Complex VIII. pDNA (45 μg, 22.5 μL of a 2 μg/μL solution in water) was added to 11.25 mL of Solution B. To this solution was added Histone H1 (36 μg, 3.6 μL of a 10 mg/mL solution in water), and the sample was mixed.
Complex IX. The lipid DOTAP-Chloride (225 μg, 9 μL of a 25 mg/mL solution in chloroform, Avanti Polar Lipids) and the lipid DOPE (225 μg, 9 μL of a 25 mg/mL solution in chloroform, Avanti Polar Lipids) were added to 500 μL of chloroform. The solution was concentrated under a stream of N₂ into a film, and dried for 16 hrs under vacuum. The film was hydrated with 11.25 mL of Solution A for 5 min, and sonicated for 20 min. pDNA (45 μg, 22.5 μL of a 2 μg/μL solution in water) was added to the mixture, and the sample was mixed for 5 min on a vortexer.
Complex X. The lipid DOTAP-Chloride (225 μg, 9 μL of a 25 mg/mL solution in chloroform, Avanti Polar Lipids) and the lipid DOPE (225 μg, 9 μL of a 25 mg/mL solution in chloroform, Avanti Polar Lipids) were added to 500 μL of chloroform. The solution was concentrated under a stream of N₂ into a film, and dried for 16 hrs under vacuum. The film was hydrated with 11.25 mL of Solution B for 5 min, and sonicated for 20 min. pDNA (45 μg, 22.5 μL of a 2 μg/μL solution in water) was added to the mixture, and the sample was mixed for 5 min on a vortexer

Tail vein injections of 1.0 mL per 10 g body weight were preformed on ICR mice (n=2) using a 30 gauge, 0.5 inch needle. Injections were done manually with injection times of 4-5 sec [Zhang et al. 1999; Liu et al. 1999]. One day after injection, the livers were harvested and homogenized in lysis buffer (0.1% Triton X-100, 0.1 M K-phosphate, 1 mM DTT, pH 7.8). Insoluble material was cleared by centrifugation and 10 μl of the cellular extract or extract diluted 10× was analyzed for luciferase activity as previously reported [Wolff et al 1990]. The results show that cationic polymer/DNA complexes were more efficiently delivered to liver cells when the complexes are injected in Solution A, relative to Solution B. Conversely, anionic polymer/pDNA complexes were more efficiently delivered to liver cells when the complexes are injected in Solution B, relative to Solution A. Cationic liposomes with pDNA were more efficiently delivered to liver cells when injected with Solution A relative to Solution B (Table 4).

TABLE 5
Nucleic acid delivery to liver.

| Com- | Luciferase Activity (RLUs) | | | |
|---|---|---|---|---|
| plex | n1 | n2 | n3 | n4 |
| Com- | 250,254,200 | 1,911,573,000 | 1,315,766,100 | |
| plex I | | | | |
| Com- | 1,294,448,100 | 1,304,320,300 | 15,330,902 | 1,713,994,600 |
| plex | | | | |
| II | | | | |
| Com- | 1,040,996,600 | 221,108,100 | 1,399,596,800 | |
| plex | | | | |
| III | | | | |
| Com- | 612,352,500 | 505,715,400 | 325,778,000 | 667,218,300 |
| plex | | | | |
| IV | | | | |
| Com- | 2,043,992,000 | 1,073,708,500 | 349,158,900 | 776,722,000 |
| plex | | | | |
| V | | | | |
| Com- | 95,870,500 | 10,643,600 | 578,100 | 1,930,400 |
| plex | | | | |
| VI | | | | |
| Com- | 49,343,900 | 38,798,800 | 29,196,500 | 16,183,100 |
| plex | | | | |
| VII | | | | |
| Com- | 1,992,733,600 | 585,884,300 | 1,339,022,600 | 1,395,211,400 |
| plex | | | | |
| VIII | | | | |
| Com- | 408,356,100 | 1,708,282,800 | 1,396,587,200 | 1,853,258,400 |
| plex | | | | |
| IX | | | | |
| Com- | 7,042,300 | 1,085,700 | 632,200 | 2,852,900 |
| plex | | | | |
| X | | | | |

### Example 7

Delivery of Plasmid DNA to Liver Cells Via Injection Into the Bile Duct Vessel:

Retrograde injection was used to deliver nucleic acid expression cassettes to hepatocytes in mouse, rat, and dog. Repetitive injections of a therapeutic gene into the bile duct were also accomplished.

The pCILuc plasmid expresses a cytoplasmic luciferase from the human CMV immediately early (hCMV ID) promoter. pCILux expresses peroxisomal luciferase under control of the hCMV IE promoter. pCILacZ plasmid expressed the β-galactosidase gene. The pCMVGH expresses human growth hormone.

Plasmid delivery into the hepatic vessels was performed in 6 week old ICR mice, 2.5 6.25 month old, 200-300 gram Sprague Dawley rats, and beagle dogs. Ventral midline incisions were performed to expose the liver and associated vessels. The mice were anesthetized with intramuscular injections of 1000 μg of ketamine HCl (Parke Davis, Morris Plains, N.J.) and by inhalation of methoxyflurane (Pitman Moore, Mudelein, Ill.) as needed. The rats were anesthetized with ether and the dogs were anesthetized with halothane by inhalation. Plasmids were injected in solutions containing 2.5 units/ml or heparin (Qian et al. 1991; Lypho Med, Inc., Chicago, Ill.) and either normal saline (0.9% NaCl) or 15% mannitol in normal saline (Sigma Chemical Co., St. Louis, Mo.).

Bile duct injections in mice were performed using manual injections with a 30-gauge, 1/2 inch needle and 1 ml syringe. A 5×1 mm, Kleinert Kutz microvessel clip was used to occlude the bile duct downstream from the point of injection in order to prevent flow to the duodenum and away from the liver. The gallbladder inlet was not occluded. In some of the bile duct injections, the junction of the hepatic vein and caudal vena cava clamped as above. In yet other injections, the portal vein and hepatic artery were clamped in addition to the occlusion of the hepatic vein. Repetitive injections into the bile duct were done by placing a polyethylene tube (I.D. 0.28 mm, O.D. 0.61 mm; Intramedic Clay Adams Brand, Becton Dickinson Co., Sparks, Md. USA) catheter into the bile duct after making a hole with a 27 gauge needle. The tubing was secured by a suture around the bile duct and tubing; thereby occluding the bite duct. The other end of the tubing was placed outside the skin of the animal's back so that surgery was not required for repeat injections. No blood vessel occlusions were done for these repetitive administrations. After completion of the studies, anatomical examination indicated that the catheter remained in the bile duct. In rats, bile duct injections were done as in mice. For the bile duct injections in dog, a suture was applied to transiently occlude the bile duct downstream from the point of injection. A DeBakey multipurpose vascular clamp was applied to the cystic duct during injection to prevent the injection solution from entering the gallbladder.

One day after injections, the animals were sacrificed and the rodent livers were divided into 6 sections composed of right lateral lobe, caudate lobe, two pieces of median lobe and two pieces of left lateral lobe. Mouse liver sections were added to 0.7 ml lysis buffer (0.1% Triton X-100, 0.1 M potassium phosphate, 1 mM DTF pH 7.8). For rats, liver sections were added to 4 ml lysis buffer. For the dog livers, approximately 10% of each lobe was divided into 5-20 pieces and placed into 2 ml lysis buffer. The samples were homogenized using a PRO 200 homogenizer (PRO Scientific Inc., Monroe, Conn.) and centrifuged at 4,000 rpm for 10 min at 4° C. 20 μl supernatant was analyzed for luciferase activity. Relative light units (RLU). were converted to pg of luciferase using standards from Analytic Luminescence Laboratories (ALL, San Diego, Calif.). Luciferase protein (pg)=5.1×10⁵×RLU+3.683 (r²=0.992).

For mouse bile ducts injected with 100 μg pCILuc in 1 ml 15% mannitol+2.5 units heparin/ml in normal saline solution, mean total luciferase protein/liver of 15.39 μg/liver was obtained when the hepatic vein was clamped. A mean total luciferase protein/liver of 1.33 μg/liver was obtained without occluding the hepatic vein. If mannitol was omitted then the bile duct injections without clamping any blood vessels yielded approximately 15-fold less luciferase (0.086 μg/liver+0.06, n=25). Clamping the hepatic artery and portal vein in addition to the hepatic vein did not improve expression beyond what was obtained when only the hepatic vein was clamped (data not shown). In rat, injections of 750 μg of pCILuc in 5-8 ml without any outflow obstruction yielded an average of 1.3 μg of luciferase/liver.

In dogs, 20 mg pCILux in 200 ml injection solution was injected at a rate of 66 mumin into the bile duct without blocking outflow by occluding the IVC. Luciferase expression was found to be evenly distributed throughout the liver. Total LUX protein in the liver calculated to be 2.96 μg.

10 μm thick tissue sections were stained for β-galactosidase expression using 1-4 hour Xgal incubations (Budker, et al., 1996). Hematoxylin was used for the counterstain but the alkaline step was omitted so that the hematoxylin stain remained red. The percent of blue stained cells in the liver sections was determined by counting ˜3000 cells in three sections and averaging. Delivery of β-galactosidase expression vector was observed in 5-10% of hepatocytes. The percent of cells stained positive for β-galactosidase correlated with the levels of luciferase expression.

Serum ALT and GGT assays were performed on mice one and eight days after each of the above injections with pCILuc (4 mice for each condition). No increases in GOT were observed. Serum ALT levels increased to 200-400 U/L one day after bile duct injections. Eight days after injection, serum ALT levels decreased to baseline levels in all animals.

Repeat Bile Duct Injections: The bile ducts of mice were cannulated and 100 μg of pCMVhGH in 1 ml of 15% mannitol in normal saline were injected once a week. Serum levels of hGH increased one day after injection and then decreased to background levels by seven days after injection. One day after the second injection, hGH levels again increased and then were back to background levels by seven days after the second injection. Only minimal increases in hGH levels occurred after the third injection. Mice that had the highest levels after the first injection had the lowest levels after the second injection (mice 3 and 6) and vice versa (mice 1, 2, and 4). In another set of animals (4 mice), the bile duct injections were repeated four times with pCMVhGH and then pCILuc was injected. The first three pCMVhGH injections led to similar increases in hGH serum levels. Although there were only minimal raises in hGH serum levels following the fourth injection, injection of pCILuc yielded an average of 29.2 ng/liver (±7.1, n=3). The liver in one of the four mice was grossly yellow and scarred as a result of the bile duct ligation and did not express any luciferase. The decrease in hGH expression following repeat procedures is presumed to result from immune response since the same animals expressed luciferase following pCILuc delivery. These results demonstrate efficient plasmid delivery following injection of expression vector in solution into the bile duct in mice, rat and dog. Occlusion of other vessels to restrict outflow of the injection solutions enhanced but was not critical for efficient expression. Expression of luciferase or β-galactosidase was evenly distributed throughout the entire liver. Furthermore, these results demonstrate the utility of the invention for use in repeat delivery. High luciferase expression was observed after a fourth delivery procedure. Such repeat delivery procedure would be useful for the treatment of genetic disorders such as hemophilia. The bile duct could be accessed repeatedly by upper gastrointestinal endoscopy. Similarly, the hepatic vein could be non-invasively accessed via peripheral or central veins. In addition, gene transfer could be delivered to newborns via the umbilical cord vessels to get them over a newborn metabolic crisis as occurs in the organic acidunas and the urea cycle defects.

### Example 8

Delivery of DNA/Polycation Complexes to Prostate and Testis Via Injection Into Dorsal Vein of Penis:

DNA and L-cystine-1,4-bis(3-aminopropyl)piperazine cationic copolymer were mixed at a 1:1.7 wt:wt ratio in water, diluted to 2.5 ml with Ringers solution and injected rapidly into the dorsal vein of the penis (within 7 seconds). For directed delivery to the prostate, clamps were applied to the inferior vena cava and the anastomotic veins just prior to the injection and removed just after the injection (within 5-10 seconds). Mice were sacrificed 24 h after injection and various organs were assayed for luciferase expression. The results, Table 4, show efficient and functional delivery of DNA containing complexes to prostate, testis and other tissues.

TABLE 6
Delivery of DNA containing plasmid to prostate
and testis via injection in dorsal vein.

| Organ | Luciferase (RLUs) |
|---|---|
| Prostate | 129,982,450 |
| Testis | 4,229,000 |
| fat (around bladder) | 730,300 |
| bladder | 618,000 |

### Example 9

Plasmid DNA Delivery to Heart Muscle Cells Via Catheter Mediate Coronary Vein Injection:

30-50 kg Yorkshire domestic swine (Sus scrofa) were sedated with telezol (20-30mg IM), induced with pentobarbitol (250-500 mg IV), and endotracheally intubated. Anesthesia was maintained with inhaled isoflurane (0.5-3%). The right carotid artery and internal jugular vein were exposed by surgical cutdown and coronary angiography was performed. Heparin (100 U/kg, IV) was administered. A 10 Fr guiding catheter was advanced to the coronary sinus, and a 7 Fr balloon-tipped triple lumen catheter was advanced over a 0.014 inch guidewire into the cardiac vein draining the left anterior descending (great cardiac vein) or right posterior descending (middle cardiac vein) territories. Injections of diluted iodinated contrast were used, in conjunction with the coronary angiogram, to delineate the myocardial territory drained by each vein.

The larger lumen of the balloon-tipped triple lumen catheter was used for fluid injection, while the smaller lumen was used to monitor cardiac vein pressures during plasmid DNA infusion. The third lumen was used to inflate and deflate the balloon. Following placement of the catheter, the balloon was inflated, and 6 ml saline or 6 ml saline with 3 mg papaverine was instilled through the large lumen (which opened distal to the balloon). The installation required 3-20 seconds and resulted in slightly increased venous pressure (10-350 mm Hg). After 5 minutes, the balloon was deflated for 20-30 seconds and then inflated again followed by injection solution delivery. A saline solution containing 100 μg/ml pCI-Luc⁺ was rapidly delivered through the main lumen. 25-30 ml injection solution was injected in 8-20 seconds. Intravenous pressure increased (120-500 mmHg). In some pigs, two sites were injected (one in the posterior descending, the other in the left anterior descending territory); in other pigs, only one site was injected (left anterior descending).

Two days following injection, the animals were sacrificed, the heart excised, divided in 1-2 gram sections, and assayed for reporter gene expression. Expression levels varied from 1.4 to 456.9 ng luciferase per gram of heart tissue (n=8).

### Example 10

Delivery of Polynucleotide to the Diaphragm in Monkey:

The monkey was anesthetized with ketamine followed by halothane inhalation. A 2 cm long incision was made in the upper thigh close to the inguinal ligament just in front of the femoral artery. Two clamps were placed around the femoral vein after separating the femoral vein from surrounding tissue. At an upstream location, the femoral vein was ligated by the clamp and a guide tube was inserted into the femoral vein anterogradely. A French 5 balloon catheter (D 1.66 mm) with guide wire was inserted into the inferior vena cava through the guide tube and an X-ray monitor was used for instructing the direction of guide wire. The guide wire was directed into the inferior phrenic vein. The catheter position in the inferior phrenic vein was checked by injecting iodine. The balloon was inflated to block blood flow through the inferior phrenic vein. 20 ml 0.017% papaverine in normal saline was injected. 5 minutes after papaverine injection, 40 ml of DNA solution (3 mg) was injected in 65 sec (0.615 ml/sec). 2 minutes after DNA injection, the balloon was released and the catheter was removed. The animal was sacrificed and the diaphragm was taken for luciferase assay 7 days after the procedure. The results indicate successful delivery of plasmid DNA to the portion of the diaphragm supplied by the injected vessel.

TABLE 7
Luciferase expression in diaphragm from monkey
sacrificed 7 days after injection of pCI-Luc⁺.

| | total luciferase | ng luciferase/ |
|---|---|---|
| diaphragm section | (ng) | gram if tissue |
| anterior part of left side | 0 | 0 |
| posterior part of left side | 0 | 0 |
| left conjunction area | 0 | 0 |
| anterior part of right side | 221.94 | 27.88 |
| posterior part of right side | 15.98 | 2.12 |
| right conjunction area | 34.21 | 17.82 |

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A process for delivering a polynucleotide to an extravascular parenchymal cell in a limb of a mammal in vivo, comprising:
a) forming an occlusion of fluid flow from said limb;
b) inserting a viral vector in a large volume into the lumen of a vessel in said limb thereby forcing fluid out of the limb vasculature and into the extravascular space and delivering said viral vector to said extravascular parenchymal cell; and,
c) releasing said occlusion.

2. The process of claim 1 wherein the viral vector is selected from the group consisting of: virus, virally encapsulated polynucleotide, and virally associated polynucleotide.

3. The process of claim 1 wherein the polynucleotide is selected from the group consisting of RNA and DNA.

4. The process of claim 2 wherein the viral vector is selected from the group consisting of: adenovirus, adeno-associated virus, retrovirus, herpes simplex virus (HSV), vaccinia virus, vesicular stomatitis virus, retrovirus, lentivirus, human immunodeficiency virus, murine leukaemia virus, and syndbis virus.

5. The process of claim 1 wherein the vessel consists of a blood vessel.

6. The process of claim 5 wherein the blood vessel consists of an artery.

7. The process of claim 5 wherein the artery is selected from the list consisting of: hepatic artery, femoral artery, iliac artery, and coronary artery.

8. The process of claim 5 wherein the blood vessel consists of a vein.

9. The process of claim 8 wherein the vein is selected from the list consisting of: portal vein, hepatic vein, tail vein, coronary vein, inferior phrenic vein and saphenous vein.

10. (canceled)

11. The process of claim 1 wherein said volume is iniected rapidly.

12. The process of claim 1 further comprising injecting a vasodilator into said limb.

13. The process of claim 1 where the parenchymal cell consists of a skeletal muscle cell.

14. (canceled)

15. A process for extravasation of a viral vector in a limb of a mammal in vivo, comprising:
a) forming an occlusion of fluid flow from said limb;
b) inserting the viral vector in a large volume into the lumen of a vessel in the limb wherein the volume of the solution and the rate of solution injection result in increased extravascular fluid volume; and,
c) removing said occlusion within two minutes of said inserting.

16. The process of claim 15 further comprising a vasodilator into the lumen of said vessel.

17. (canceled)
